(19) United States
(12) Patent Application Publication
(10) Pub. No.: US 2026/0305938 A1
(43) Pub. Date: Oct. 8, 2026

(54) IMAGE PROCESSING DEVICE, IMAGE PROCESSING METHOD, AND STORAGE MEDIUM

(71) Applicant: NEC Corporation, Tokyo (JP)

(72) Inventors: Terumi UMEMATSU, Tokyo (JP); Yoshifumi ONISHI, Tokyo (JP)

(73) Assignee: NEC Corporation, Tokyo (JP)

(21) Appl. No.: 19/164,398

(22) PCT Filed: Mar. 27, 2023
(86) PCT No.: PCT/JP2023/012153

§ 371 Date: Sep. 11, 2025

(51) Int. Cl. A61B 3/113; A61B 3/11

(52) U.S. Cl. CPC A61B 3/113; A61B 3/112

(57) ABSTRACT

A first instruction means 14X of an image processing device instructs to be in a first state in which a subject is looking at a position ahead by a first distance. A first generation means 15X generates first pupil information relating to the subject's pupil based on an image generated by a photographing means in the subject's first state. A steadiness assessment means 16X determines presence or absence of steadiness of the subject's pupil in the first state based on the first pupil information. A second instruction means 17X instructs, upon determining the presence of the steadiness, to be in a second state in which the subject is looking at a position ahead by a second distance shorter than the first distance. A second generation means 18X generates second pupil information relating to the pupil based on an image generated by the photographing means in the subject's second state.

### TECHNICAL FIELD

The present disclosure relates to a technical field of an image processing device, an image processing method, and a storage medium that perform processing relating to measurement of a near view reaction by an image.

### BACKGROUND

A system that measures a pupil of a subject based on a face image obtained by photographing the subject is known. For example, PTL 1 discloses a system that detects a region of a pupil of a subject based on video signals from two cameras and analyzes a pupil area, a pupil diameter, and a position of the pupil.

### CITATION LIST

### Patent Literature

PTL 1: WO 2002/003853 A1

### SUMMARY

### Problem to be Solved

In general, a dedicated device or system has been required for measuring a near view reaction. On the other hand, in a case where measurement regarding a near view reaction is routinely performed for the purpose of self-care or the like, it is desirable that the subject himself/herself can easily perform the measurement.

In view of the above-described problems, an object of the present disclosure is to provide an image processing device, an image processing method, and a storage medium capable of suitably generating information relating to a near view reaction of a subject from an image obtained by photographing the subject.

### Means for Solving the Problem

In one mode of the image processing device, an image processing device includes:
a first instruction means for instructing a subject to be in a first state in which the subject is looking at a position ahead by a first distance;
a first generation means for generating first pupil information that is information relating to a pupil of the subject based on an image generated by a photographing means in a case where the subject is in the first state;
a steadiness assessment means for determining presence or absence of steadiness of the pupil of the subject in the first state based on the first pupil information;
a second instruction means for instructing, upon determining the presence of the steadiness, the subject to be in a second state in which the subject is looking at a position ahead by a second distance shorter than the first distance; and
a second generation means for generating second pupil information that is information relating to the pupil based on an image generated by the photographing means in a case where the subject is in the second state.

In one mode of the image processing method, an image processing method executed by a computer includes:
instructing a subject to be in a first state in which the subject is looking at a position ahead by a first distance;
generating first pupil information that is information relating to a pupil of the subject based on an image generated by a photographing means in a case where the subject is in the first state;
determining presence or absence of steadiness of the pupil of the subject in the first state based on the first pupil information;
instructing, upon determining the presence of the steadiness, the subject to be in a second state in which the subject is looking at a position ahead by a second distance shorter than the first distance; and
generating second pupil information that is information relating to the pupil based on an image generated by the photographing means in a case where the subject is in the second state.

In one mode of the storage medium, a storage medium stores a program for causing a computer to perform a process including:
instructing a subject to be in a first state in which the subject is looking at a position ahead by a first distance;
generating first pupil information that is information relating to a pupil of the subject based on an image generated by a photographing means in a case where the subject is in the first state;
determining presence or absence of steadiness of the pupil of the subject in the first state based on the first pupil information;
instructing, upon determining the presence of the steadiness, the subject to be in a second state in which the subject is looking at a position ahead by a second distance shorter than the first distance; and
generating second pupil information that is information relating to the pupil based on an image generated by the photographing means in a case where the subject is in the second state.

### Effect

An example advantage according to the present invention is to suitably generate information relating to a near view reaction of a subject from an image obtained by photographing the subject.

### BRIEF DESCRIPTION OF THE DRAWINGS

FIG. 1 illustrates a schematic configuration of a near view reaction measurement system according to a first example embodiment.

FIG. 2 illustrates a state at the time of measuring a near view reaction in a case where the near view reaction measurement system is one terminal device.

FIG. 3 illustrates an example of a hardware configuration of an image processing device common to each example embodiment.

FIG. 4 is an example of functional blocks of an image processing device relating to near view reaction measurement processing in the first example embodiment.

FIG. 5 is an example of a camera image obtained by photographing a face of a subject.

FIG. 6 is an example of a graph illustrating a temporal change in a pupil size when a state is switched from a far view state to a near view state.

FIG. 7 is an example of a graph illustrating a temporal change of a convergence degree recognized in a case where a far view instruction is performed again after a near view instruction.

FIG. 8 is an example of a flowchart relating to near view reaction measurement processing in the first example embodiment.

FIG. 9 illustrates a schematic configuration of a near view reaction measurement system in a second example embodiment.

FIG. 10 is a block diagram of an image processing device in a third example embodiment.

FIG. 11 is an example of a flowchart executed by the image processing device in the third example embodiment.

### EXAMPLE EMBODIMENT

Hereinafter, example embodiments of an image processing device, an image processing method, and a storage medium will be described with reference to the drawings.

### First Example Embodiment

### (1) System Configuration

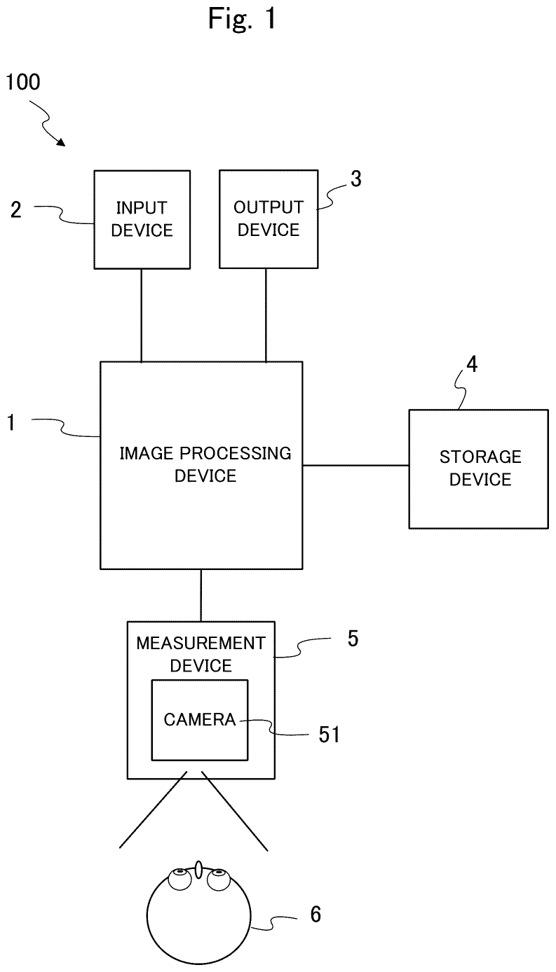

FIG. 1 illustrates a schematic configuration of a near view reaction measurement system 100 according to a first example embodiment. The near view reaction measurement system 100 is a system that simply measures a near view reaction of a subject 6 based on an image obtained by photographing the face of the subject 6 with a visible light camera, and mainly includes an image processing device 1, an input device 2, an output device 3, a storage device 4, and a measurement device 5 including a camera (imaging device) 51. For example, the subject 6 simply measures the subject's own near view reaction by the near view reaction measurement system 100 for the purpose of managing the health condition (including self-care).

The image processing device 1 measures the near view reaction of the subject 6 based on the face image (the same applies hereinafter, including a video which is a predetermined number of image sequences obtained in time series) of the subject 6 generated by the camera 51, and outputs the measurement result. The image processing device 1 performs data communication with the input device 2, the output device 3, the storage device 4, and the measurement device 5 via a communication network or by wireless or wired direct communication.

The input device 2 is an interface that receives a user input (manual input). A user who inputs the information using the input device 2 may be the subject 6 himself/herself or a person who manages or supervises activities of the subject 6. The input device 2 may be, for example, various user input interfaces such as a touch panel, a button, a keyboard, a mouse, and an audio input device. The input device 2 supplies the input signal generated based on the user input to the image processing device 1.

The output device 3 outputs predetermined information based on the output signal supplied from the image processing device 1. In this case, the output signal includes at least one of a display signal and an audio signal. Then, the output device 3 displays information based on the display signal supplied from the image processing device 1, and outputs audio of the information based on the audio signal supplied from the image processing device 1. The output device 3 includes, for example, at least one of a display device such as a display or a projector and an audio output device such as a speaker.

The storage device 4 is a memory that stores various types of information necessary for the measurement of the near view reaction and the like. The storage device 4 may be an external storage device such as a hard disk connected to or incorporated in the image processing device 1, or may be a storage medium such as a flash memory. The storage device 4 may be a server device that performs data communication with the image processing device 1. The storage device 4 may include a plurality of devices.

The measurement device 5 is one or a plurality of sensors including the camera 51 which is a visible light camera. For example, the measurement device 5 may include an illuminance sensor for detecting a light amount change of external light under a measurement environment of a near view reaction of the subject 6. The measurement device 5 supplies a signal measured by each sensor to the image processing device 1. Hereinafter, the image generated by the camera 51 is also referred to as a “camera image”. The camera 51 is an example of a “photographing means”.

The configuration of the near view reaction measurement system 100 illustrated in FIG. 1 is an example, and various modifications may be made to the configuration. For example, the image processing device 1, the input device 2, the output device 3, the storage device 4, and the measurement device 5 may be achieved by one terminal device as a smartphone, a tablet terminal, or the like.

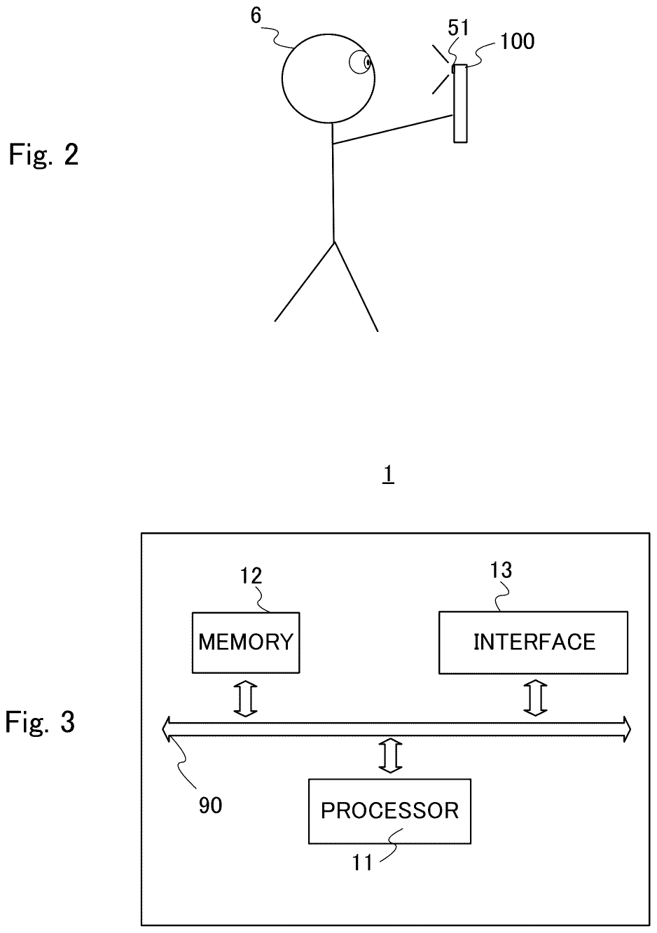

FIG. 2 illustrates a state at the time of measuring a near view reaction in a case where the near view reaction measurement system 100 is one terminal device (for example, a smartphone). As illustrated in FIG. 2, the subject 6 grips the near view reaction measurement system 100 which is a terminal device, and adjusts the orientation of the terminal device such that the subject's face is included in the photographing range of the camera 51. The near view reaction measurement system 100 may be fixed to a tripod or the like. Then, in the state illustrated in FIG. 2, the subject 6 sequentially performs far view and near view according to an instruction (guidance) output by the near view reaction measurement system 100, and causes the near view reaction measurement system 100 to measure the near view reaction.

### (2) Hardware Configuration

FIG. 3 illustrates the hardware configuration of the image processing device 1. The image processing device 1 includes, as hardware, a processor 11, a memory 12, and an interface 13. The processor 11, the memory 12, and the interface 13 are connected via a data bus 90.

The processor 11 functions as a controller (arithmetic device) that takes overall control of the image processing device 1 by executing a program stored in the memory 12. The processor 11 is, for example, a processor such as a central processing unit (CPU), a graphics processing unit (GPU), or a tensor processing unit (TPU). The processor 11 may include a plurality of processors. The processor 11 is an example of a computer.

The memory 12 includes various volatile memories and nonvolatile memories, such as a random access memory (RAM), a read only memory (ROM), and a flash memory. The memory 12 stores a program for executing processing to be performed by the image processing device 1. A part of the information stored in the memory 12 may be stored in one or more external storage devices capable of communicating with the image processing device 1, or may be stored in a storage medium detachable from the image processing device 1. The memory 12 may function as at least a part of the storage device 4.

The interface 13 is an interface for electrically connecting the image processing device 1 and another device. These interfaces may be a wireless interface such as a network adapter for wirelessly transmitting and receiving data to and from the another device, or may be a hardware interface for connecting to the another device by a cable or the like.

The hardware configuration of the image processing device 1 is not limited to the configuration illustrated in FIG. 3. For example, the image processing device 1 may include at least one of the input device 2, the output device 3, the storage device 4, and the measurement device 5.

### (3) Outline of Near View Reaction Measurement Processing

Next, a near view reaction measurement processing which is processing relating to the measurement of a near view reaction will be described. Schematically, the image processing device 1 determines whether the pupil of the subject 6 is in a steady state based on a camera image generated after the subject 6 is instructed to perform far viewing. Then, in a case where the pupil of the subject 6 is in a steady state, the image processing device 1 instructs the subject 6 to perform near viewing, and calculates an index regarding a near view reaction based on information relating to the time-series pupil of the subject 6 recognized from the camera image generated after the instruction. As a result, the image processing device 1 can suitably measure the near view reaction based on the camera image.

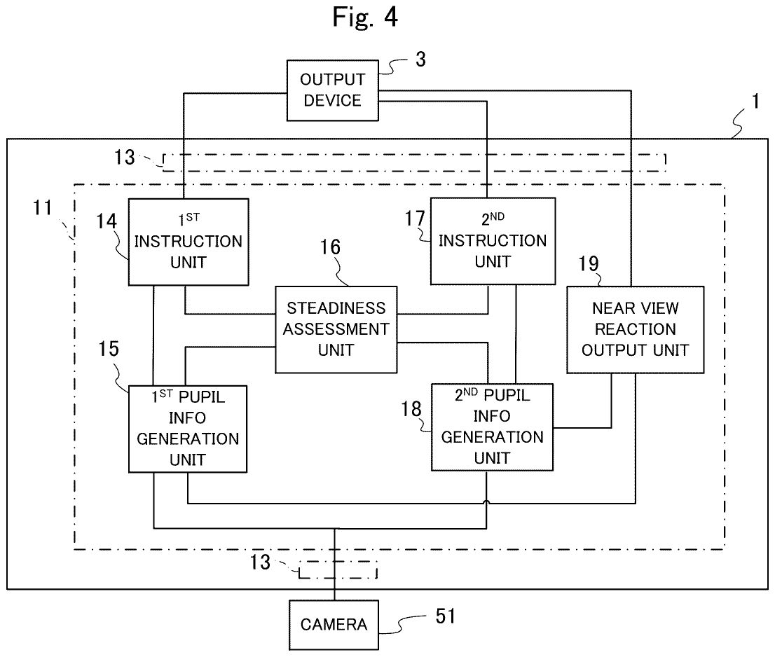

FIG. 4 is an example of functional blocks of the image processing device 1 relating to the near view reaction measurement processing. The processor 11 of the image processing device 1 functionally includes a first instruction unit 14, a first pupil information generation unit 15, a steadiness assessment unit 16, a second instruction unit 17, a second pupil information generation unit 18, and a near view reaction output unit 19. In FIG. 4, blocks between which data is exchanged are connected by a solid line, but a combination of the blocks between which the data is exchanged is not limited to the illustrated combination. The same applies to diagrams of other functional blocks described later.

In a case where it is determined that the measurement of the near view reaction should be started, the first instruction unit 14 issues an instruction (also referred to as a “far view instruction”) to the subject 6 so that the subject 6 is in the far view state. Specifically, the first instruction unit 14 instructs the subject 6 to focus on a place a predetermined distance (also referred to as a “first distance”) ahead. The “first distance” is a focal length at which the subject 6 can be regarded as a far view state, and is, for example, a distance equal to or more than 6 m at which the focal length is substantially infinite. Therefore, for example, the first instruction unit 14 generates an output signal including at least one of a display signal or an audio signal instructing to focus on a location equal to or more than 6 m ahead, and supplies the output signal to the output device 3 via the interface 13. As a result, the first instruction unit 14 causes the output device 3 to output the far view instruction to the subject 6. In the case of outputting the far view instruction, the first instruction unit 14 notifies the first pupil information generation unit 15 that the far view instruction has been output.

Here, the determination as to whether to start the measurement of the near view reaction will be supplementarily described. For example, in a case where a predetermined user input by the input device 2 instructing the start of the near view reaction measurement is detected, the first instruction unit 14 determines that the near view reaction measurement should be started. In another example, the first instruction unit 14 determines that the measurement of the near view reaction should be started at the measurement timing of the near view reaction planned in advance. In this case, the information relating to the measurement timing is stored in advance in the storage device 4 or the memory 12.

The first pupil information generation unit 15 generates information relating to the pupil of the subject 6 based on the camera image acquired from the camera 51 via the interface 13 during a period from the output of the instruction by the first instruction unit 14 to the determination that the pupil of the subject 6 has steadiness by the steadiness assessment unit 16. In the above-described period, the first pupil information generation unit 15 considers that the subject 6 is looking at a place where the focal length becomes the first distance (for example, infinity) (that is, it is a far view state and is also referred to as a “first state”), and generates information relating to the pupil of the subject 6 in the far view state. The information relating to the pupil of the subject 6 is also referred to as “pupil information”. In the present example embodiment, the first pupil information generation unit 15 calculates, as the pupil information, at least a pupil size (for example, a pupil diameter) of the subject 6 and a degree of convergence (that is, the state of the eyes) of the eyes of the subject 6 (also referred to as “convergence degree”). A specific example of the pupil diameter and the convergence degree will be described later. The first pupil information generation unit 15 supplies the pupil information (also referred to as “first pupil information”) generated by the first pupil information generation unit 15 to the steadiness assessment unit 16 and the near view reaction output unit 19.

After the first instruction unit 14 outputs the far view instruction, the steadiness assessment unit 16 determines whether the pupil of the subject 6 recognized from the camera image has steadiness based on first pupil information. In this case, the steadiness assessment unit 16 considers that the subject 6 is in the far view state after the far view instruction by the first instruction unit 14, and determines the presence or absence of the steadiness of the pupil of the subject 6 recognized in the far view state. Then, in a case where it is determined that there is steadiness, the steadiness assessment unit 16 notifies the second instruction unit 17 and the second pupil information generation unit 18 that the processing is to be started.

Here, a supplementary description will be given of the standard of the presence or absence of steadiness. For example, after the far view instruction, the steadiness assessment unit 16 determined that there is steadiness in a case where the following (a) to (c) are continuously satisfied for a predetermined time:
(a) no change in the focal point of the subject 6,
(b) no photoreaction, and
(c) the stable pupil size,

and in a case where any of the conditions (a) to (c) is not continuously satisfied for a predetermined time, it is determined that there is no steadiness. The length of the predetermined time is, for example, a predetermined value stored in advance in the storage device 4, the memory 12, or the like.

First, the condition (a) will be described. For example, the steadiness assessment unit 16 determines whether the condition (a) is satisfied based on the time-series convergence degree indicated by the pupil information supplied from the first pupil information generation unit 15 in the immediately preceding predetermined time. Then, the steadiness assessment unit 16 determines that the condition (a) is satisfied in a case where the convergence degree does not substantially change in the predetermined time, and determines that the condition (a) is not satisfied in a case where the convergence degree substantially changes in the predetermined time. In this case, for example, the steadiness assessment unit 16 determines whether the convergence degree is substantially changed based on a variance of a predetermined number of convergence degrees obtained within the predetermined time, a difference between a maximum value and a minimum value, or a comparison between a statistic indicating other variations and a predetermined threshold. The above-described threshold is stored in advance in the storage device 4, the memory 12, or the like, for example.

Next, the condition (b) will be described. For example, the steadiness assessment unit 16 determines whether the condition (b) is satisfied based on a time-series light amount (illuminance) measured by the measurement device 5 in the immediately preceding predetermined time. Specifically, the steadiness assessment unit 16 determines that the condition (b) is satisfied in a case where the illuminance measured by the measurement device 5 does not substantially change in the predetermined time described above, and determines that the condition (b) is not satisfied in a case where the illuminance measured by the measurement device 5 substantially changes in the predetermined time described above. In this case, for example, the steadiness assessment unit 16 determines whether the illuminance is substantially changed based on a variance of a predetermined number of measurement values of illuminance obtained within the predetermined time, a difference between a maximum value and a minimum value, or a comparison between a statistic indicating other variations and a predetermined threshold. The above-described threshold is stored in advance in the storage device 4, the memory 12, or the like, for example.

In another example, the steadiness assessment unit 16 may determine whether the condition (b) is satisfied based on the pupil size in time series. In this case, the condition (b) is the same as the condition (c) described later.

Next, the condition (c) will be described. For example, the steadiness assessment unit 16 determines whether the condition (c) is satisfied based on the presence or absence of a substantial change in the time-series pupil size indicated by the pupil information supplied from the first pupil information generation unit 15 in the immediately preceding predetermined time. In this case, the steadiness assessment unit 16 determines the presence or absence of a substantial change in the pupil size based on the dispersion of the predetermined number of pupil sizes based on the pupil information obtained within the above-described predetermined time, the difference between the maximum value and the minimum value, or the comparison result between the predetermined threshold and a statistic indicating other dispersion. The above-described threshold is stored in advance in the storage device 4, the memory 12, or the like, for example.

In a case where the steadiness assessment unit 16 determines that there is steadiness, the second instruction unit 17 instructs (also referred to as a “near view instruction”) the subject 6 so that the subject 6 is in a near view state. Specifically, the second instruction unit 17 instructs the subject 6 to focus on a place a predetermined distance (also referred to as a “second distance”) ahead. The above-described “second distance” is a focal length at which the subject 6 can be regarded as being in a near view state, and is a distance (for example, a distance of less than 50 cm) shorter than the first distance. For example, in the configuration illustrated in FIG. 2 (that is, the configuration in which the subject 6 grips the terminal device which is the near view reaction measurement system 100), the second instruction unit 17 generates an output signal including at least one of a display signal or an audio signal for instructing focusing on the camera 51 provided in the terminal device gripped by the subject 6, and supplies the output signal to the output device 3 via the interface 13. As a result, the first instruction unit 14 causes the output device 3 to output the near view instruction to the subject 6. In the case of outputting the near view instruction, the first instruction unit 14 notifies the first pupil information generation unit 15 that the near view instruction has been output.

The second pupil information generation unit 18 generates pupil information in time series of the subject 6 based on the camera image acquired from the camera 51 via the interface 13 during a period until a predetermined time (for example, several seconds) elapses after the second instruction unit 17 outputs the near view instruction. In the above-described period, the first pupil information generation unit 15 considers that the subject 6 is looking at a place where the focal length becomes the second distance (that is, it is a near view state and is also referred to as a “second state”), and generates the pupil information including the pupil size and the convergence degree in time series of the subject 6 in the near view state. The second pupil information generation unit 18 supplies the pupil information (the second pupil information is also referred to as “second pupil information”) generated by the second pupil information generation unit 18 to the near view reaction output unit 19.

The near view reaction output unit 19 calculates an index relating to the near view reaction (also referred to as a “near view reaction index”) based on the first pupil information supplied from the first pupil information generation unit 15 and the second pupil information supplied from the second pupil information generation unit 18, and causes the output device 3 to display or output audio of information relating to the calculation result of the near view reaction index. In this case, the near view reaction output unit 19 calculates the near view reaction index based on the first pupil information (that is, the pupil information in the far view state at the time point when the steadiness assessment unit 16 determines that there is the steadiness) most recently supplied from the first pupil information generation unit 15 and the time-series second pupil information supplied from the second pupil information generation unit 18. The type of the near view reaction index to be calculated differs depending on the application to which the near view reaction measurement system 100 is applied, and specific examples thereof will be described later in the sections of “(4) Pupil Information and near view reaction index” and “(7) Application”.

Here, the information relating to the calculation result of the near view reaction index that the near view reaction output unit 19 outputs to the output device 3 may be information indicating the calculated near view reaction index itself, or may be information relating to the state of the subject 6 or the like estimated based on the near view reaction index. The latter example will be specifically described in the section of “(7) Application”. The near view reaction output unit 19 generates an output signal (that is, at least one of a display signal and an audio signal) for outputting information relating to the calculation result of the calculated near view reaction index, and supplies the output signal to the output device 3 via the interface 13. As a result, the near view reaction output unit 19 causes the output device 3 to output information relating to the calculation result of the near view reaction index.

The near view reaction output unit 19 may calculate the near view reaction index based on the time-series second pupil information without using the first pupil information. In this case, the second pupil information obtained first in time series is regarded as the pupil information in the far view state, and the above-described near view reaction index is calculated.

The image processing device 1 may calculate the near view reaction index for a predetermined number of times by executing the processing of the first instruction unit 14, the first pupil information generation unit 15, the steadiness assessment unit 16, the second instruction unit 17, the second pupil information generation unit 18, and the near view reaction output unit 19 a predetermined number of times. In this case, the near view reaction output unit 19 calculates a time average or another representative value of the near view reaction indexes for a predetermined number of times, and outputs the calculation result to the output device 3. As a result, it is possible to output the highly accurate near view reaction index subjected to the statistical processing.

The near view reaction output unit 19 may further calculate an index (also referred to as a “far view reaction index”) indicating the reaction of the subject 6 accompanying the switching from the near view state to the far view state. In this case, after the near view instruction by the second instruction unit 17, the first instruction unit 14 again performs the far view instruction, and the near view reaction output unit 19 calculates the far view reaction index based on the time-series first pupil information (and the immediately preceding second pupil information) generated by the first pupil information generation unit 15 after the far view instruction. This specific example will be described in the section of “(7) Application”.

The components of the first instruction unit 14, the first pupil information generation unit 15, the steadiness assessment unit 16, the second instruction unit 17, the second pupil information generation unit 18, and the near view reaction output unit 19 described in FIG. 4 can be achieved, for example, by the processor 11 executing a program. Each component may also be achieved by recording a necessary program in an optional nonvolatile storage medium and installing the program as necessary. At least a part of these components is not limited to be achieved by software by a program, and may be achieved by a combination of any of hardware, firmware, and software, or the like. At least a part of these components may be achieved using, for example, a user-programmable integrated circuit such as a field-programmable gate array (FPGA) or a microcontroller. In this case, a program including the above components may be achieved by using the integrated circuit. At least a part of the components may include an application specific standard produce (ASSP), an application specific integrated circuit (ASIC), or a quantum processor (quantum computer control chip). In this manner, the components may be achieved by various types of hardware. The same applies to other example embodiments described later. These components may also be achieved by, for example, cooperation of a plurality of computers by using a cloud computing technology or the like.

(4) Pupil information and Near View Reaction Index

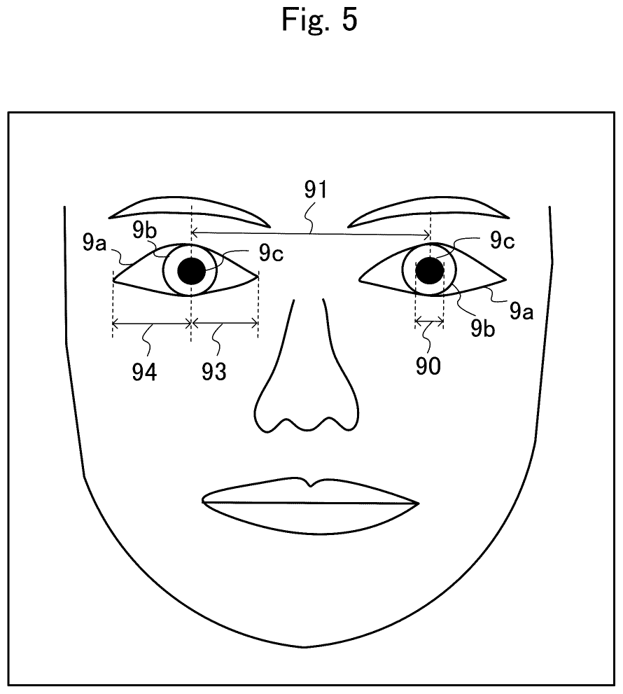

First, a specific example of the pupil information will be described. FIG. 5 is an example of a camera image obtained by photographing the face of the subject 6.

The image processing device 1 recognizes each region of the eye (both eyes in this case) 9a, an iris 9b, and a pupil 9c of the subject 6 from a camera image obtained by photographing the face of the subject 6 based on an arbitrary image recognition technology. Then, the image processing device 1 calculates pupil information indicating a pupil size and a convergence degree to be described later based on each recognized region.

For example, the image processing device 1 calculates a pupil size (that is, the pupil diameter) relevant to the length of the arrow 90. The image processing device 1 may calculate the pupil sizes of both eyes and further generate pupil information indicating an average of the calculated pupil sizes.

In another example, the image processing device 1 calculates the pupillary distance relevant to the length of the arrow 91 as an example of the convergence degree. As described above, the image processing device 1 can recognize accurate convergence by calculating the convergence degree based on both eyes. In this case, the camera image is an image obtained by photographing at least both eyes of the subject 6, and the image processing device 1 calculates the convergence degree based on the positions of the pupils 9c of both eyes in the camera image. The image processing device 1 may calculate the pupil information based on a camera image obtained by photographing only one eye of the subject. In this case, for example, the image processing device 1 may calculate the relative position (that is, a distance relevant to the lengths of the arrows 93 and 94) of the center of the pupil 9c with respect to both ends of the eye 9a as the convergence degree. In this manner, the image processing device 1 may calculate a value indicating the relative position of the pupil 9c in the eye 9a as the convergence degree.

Here, the image processing device 1 may perform normalization processing of converting the pupil size, the pupillary distance, and the like from a size based on the number of pixels on the camera image to a size of a predetermined scale (for example, an actual size), and generate pupil information indicating the normalized pupil size and the pupillary distance. In this case, for example, the image processing device 1 may normalize the pupil size and the pupillary distance based on the size of the iris 9b by using the characteristics that the size of the iris 9b has a small individual difference and does not change over time. In this case, for example, information indicating the relationship between the size of the iris 9b on the image and the size of the iris 9b after normalization is stored in advance in the storage device 4, the memory 12, or the like, and the image processing device 1 performs the above-described normalization with reference to this information. In another example, in a case where a plurality of cameras having different viewpoints are provided as the camera 51 in the near view reaction measurement system 100, the image processing device 1 may recognize the actual size of the pupil size and the pupillary distance by three-dimensionally restoring the face of the subject 6 from the camera images of the plurality of cameras based on an arbitrary three-dimensional restoration technique such as SfM (Structure From Motion).

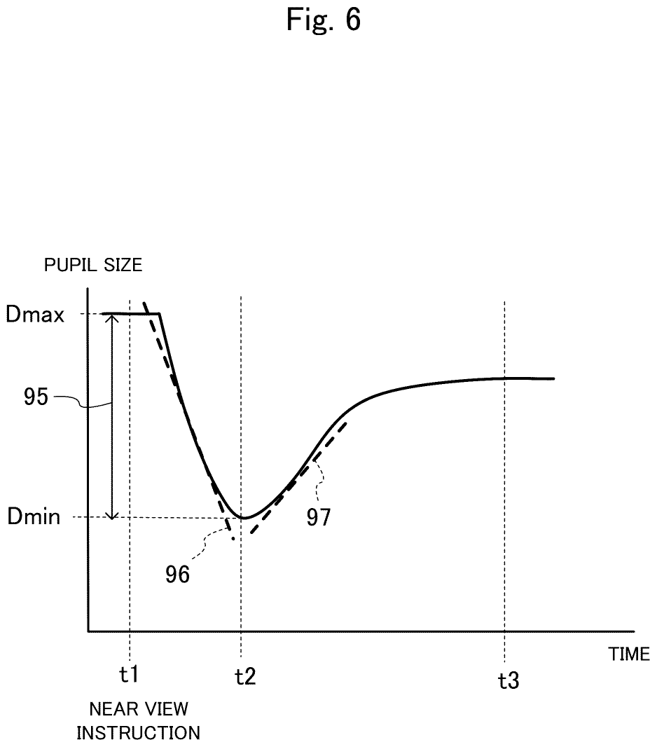

Next, a specific example of the near view reaction index will be described. First, the near view reaction index obtained from the pupil size will be described. FIG. 6 is an example of a graph illustrating a temporal change in the pupil size according to the near view reaction.

In this case, after the time “t1” at which the near view instruction is issued, the pupil size (here, the pupil radius) of the subject 6 gradually decreases from the maximum value “Dmax” through the latency due to the subject 6 switching from far view to near view, and becomes the minimum value “Dmin” at the time “t2”. Thereafter, the pupil size is enlarged again, and becomes a steady state near the time “t3” which is a predetermined time after time t2.

In this case, for example, the image processing device 1 calculates a maximum pupil contraction amount relevant to the length (that is, Dmax−Dmin) of the arrow 95, a maximum pupil contraction rate relevant to the ratio between the size Dmax and the size Dmin, a pupil contraction speed relevant to the inclination of the line 96, a re-expansion speed relevant to the inclination of the line 97, and the like as the near view reaction index. The inclination of the line 96 is relevant to the decrease speed of the pupil size in the decrease period of the pupil size, and the inclination of the line 97 is relevant to the increase speed of the pupil size in the increase (re-expansion) period of the pupil size.

The near view reaction index based on the pupil size is not limited to the above-described index, and may be any index based on a time-series pupil size measured in a period before and after switching from far view to near view. The image processing device 1 may also calculate the far view reaction index based on the pupil size in the same manner as the above-described near view reaction index.

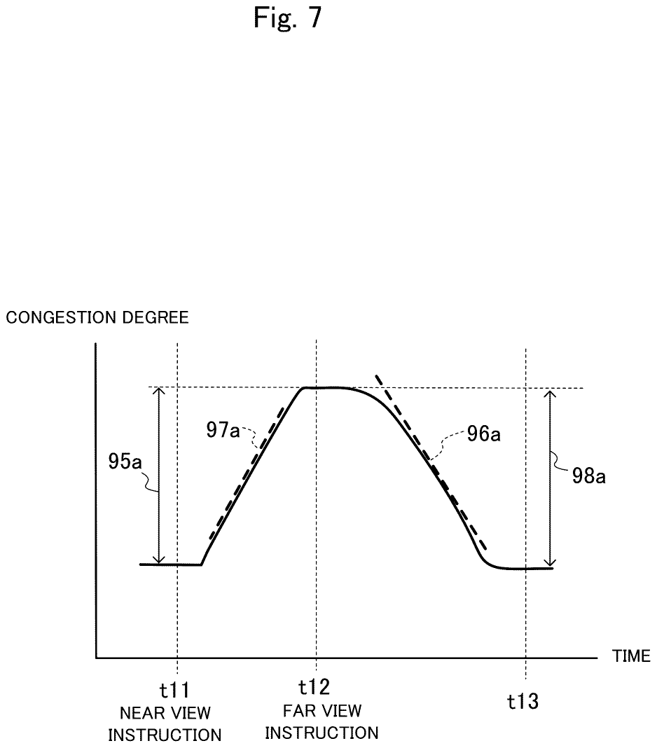

Next, the near view reaction index relating to the convergence degree will be described. FIG. 7 is an example of a graph illustrating a temporal change in the convergence degree recognized in a case where the first instruction unit 14 performs the far view instruction again after the near view instruction by the second instruction unit 17. Here, as an example, it is assumed that the higher the convergence degree, the larger the value.

In the example of FIG. 7, after the time “t11” at which the near view instruction is issued, the subject 6 switches from far view to near view, so that the convergence degree gradually increases from the minimum amount through the latency and then becomes a steady state. Further, the far view instruction is given at the time “t12” when the steady state is reached, and the convergence degree gradually decreases through the latency due to the subject 6 switching from near view to far view, and becomes the steady state near the time “t13”.

In this case, for example, similarly to the example of FIG. 6, the image processing device 1 calculates, as the near view reaction index, a difference (relevant to the length of the arrow 95a) between the minimum value of the convergence degree relevant to the far view state and the maximum value of the convergence degree relevant to the near view state and an increase speed of the convergence degree (relevant to the inclination of the arrow 97a). For example, the image processing device 1 calculates, as the far view reaction index, a difference (relevant to the length of the arrow 98a) between the maximum value of the convergence degree relevant to the near view state and the minimum value of the convergence degree relevant to the far view state, and the decrease speed of the convergence degree in a decrease period of the convergence degree associated with switching from near view to far view (relevant to the inclination of the arrow 96a). Here, the difference between the minimum value and the maximum value of the convergence degree described above is a degree of change in the convergence degree, and is also referred to as a “convergence amount” hereinafter. The increase speed and the decrease speed described above are hereinafter also referred to as “change speed of the convergence degree”. The convergence amount and the change speed in the convergence degree are examples of “an index relating to a change in the degree of convergence”.

The near view reaction index based on the convergence degree is not limited to the above-described index, and may be any index based on a time-series convergence degree measured in a period before and after switching from far view to near view. Similarly, the far view reaction index based on the convergence degree is not limited to the above-described index, and may be an arbitrary index based on a time-series convergence degree measured in a period before and after switching from near view to far view.

### (5) Processing Flow

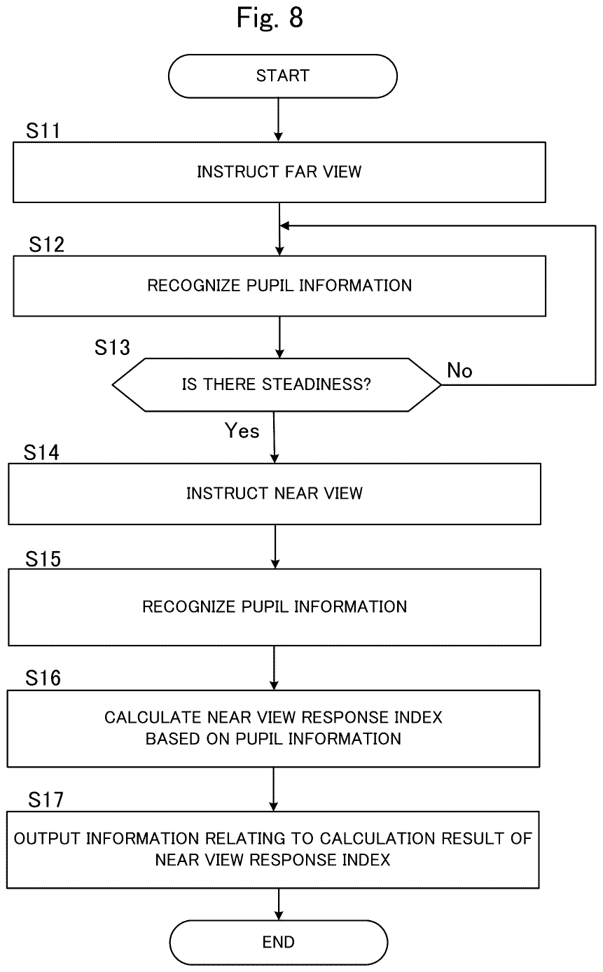

FIG. 8 is an example of a flowchart relating to the near view reaction measurement processing executed by the image processing device 1.

First, the image processing device 1 outputs a far view instruction for instructing the subject 6 to perform far viewing by the output device 3 (step S11). Then, the image processing device 1 generates pupil information (that is, the first pupil information) of the subject 6 based on each of the time-series camera images generated by the camera 51 after step S11 (step S12).

Then, the image processing device 1 determines the presence or absence of the steadiness of the pupil of the subject 6 based on the pupil information or the like obtained in step S12 (step S13). In this case, the image processing device 1 determines the presence or absence of the steadiness of the pupil based on the predetermined number of pieces of pupil information generated in the immediately preceding predetermined period in step S12. Then, in a case where the image processing device 1 determines that the pupil of the subject 6 has steadiness (step S13; Yes), the process proceeds to step S14. On the other hand, in a case where the image processing device 1 determines that the pupil of the subject 6 has no steadiness (step S13; No), subsequently in step S12, the pupil information is generated based on the latest camera image generated by the camera 51.

Next, after it is determined that the pupil of the subject 6 has steadiness, the image processing device 1 outputs a near view instruction to instruct the subject 6 to perform near viewing by the output device 3 (step S14). Then, the image processing device 1 generates pupil information (that is, the second pupil information) of the subject 6 based on each of the time-series camera images generated by the camera 51 after step S14 (step S15). Then, the image processing device 1 calculates a near view reaction index based on the pupil information (step S16). In this case, the image processing device 1 calculates one or a plurality of types of near view reaction indexes based on the time-series second pupil information obtained in step S15 (and the first pupil information obtained in step S12). The image processing device 1 may calculate a time average (or other representative value) of the near view reaction indexes for each type based on samples of the near view reaction indexes obtained by repeating steps S11 to S16 a plurality of times.

Then, the image processing device 1 outputs information relating to the calculation result of the near view reaction index by the output device 3 (step S17). In this case, the image processing device 1 may determine the state of the subject 6 regarding the presence or absence of an aging phenomenon, the presence or absence of fatigue, the presence or absence of a nervous system disorder, and the like from the calculated near view reaction index according to the application to be applied, and output the determination result by the output device 3. An example of the application will be described later.

### (6) Modifications

Next, modifications of the above-described example embodiments will be described. The following modifications may be arbitrarily combined and applied to the above-described example embodiments.

### (First Modification)

In the far view instruction and the near view instruction, the image processing device 1 may output guidance so that the distance (photographing distance) between the subject 6 and the camera 51 becomes a predetermined distance based on the size of the iris of the subject 6 recognized from the camera image.

In this case, for example, in step S11, the image processing device 1 outputs the far view instruction and recognizes the size of the iris of the subject 6 from the camera image. Then, the image processing device 1 determines whether the size of the recognized iris belongs to the appropriate size range. The appropriate size range described above is a range of the size of the iris relevant to a range of the photographing distance within a distance range preferable for the near view reaction measurement processing, and is, for example, a range stored in advance in the storage device 4, the memory 12, or the like. Then, in a case where the size of the recognized iris is out of the appropriate size range, the image processing device 1 causes the output device 3 to output information instructing the subject 6 to adjust the photographing distance. In this case, in a case where the size of the recognized iris is smaller than the appropriate size range, the image processing device 1 instructs to shorten the photographing distance, and in a case where the size of the recognized iris is larger than the appropriate size range, the image processing device 1 instructs to lengthen the photographing distance. Similarly in step S14 and the like, the image processing device 1 may recognize the size of the iris of the subject 6 from the camera image and output guidance on the photographing distance based on the determination result as to whether the recognized size of the iris belongs to the appropriate size range.

According to the present modification, the image processing device 1 can suitably adjust the photographing distance to be a distance suitable for measuring the near view reaction.

### (Second Modification)

The acquisition of the time-series second pupil information is not essential, and the image processing device 1 may acquire the second pupil information at least at one time point in the near view state.

In this case, for example, the near view reaction output unit 19 calculates the near view reaction index based on the second pupil information based on the camera image generated at the predetermined time after the far view instruction by the second instruction unit 17 and the first pupil information generated last before the far view instruction by the second instruction unit 17. The above-described predetermined time may be, for example, a time relevant to a time between time t1 and time t2 in FIG. 6 (that is, a time within the decrease period of the pupil size), or may be a time near the steady time t3 (that is, the time when the state of the pupil becomes the steady state). In the former example, the near view reaction output unit 19 can calculate the difference between the pupil size indicated by the first pupil information and the pupil size indicated by the second pupil information as the maximum pupil contraction rate. In the latter example, the near view reaction output unit 19 can calculate the difference between the pupil size indicated by the first pupil information and the pupil size indicated by the second pupil information as the difference between the pupil sizes of the near view and the far view in the steady state.

### (7) Application

Next, an application of the near view reaction measurement system 100 will be exemplified. In the following application, the near view reaction measurement system 100 estimates the state of the subject 6 (particularly, the state relating to health) based on the calculated near view reaction index, and outputs the estimation result of the state of the subject 6. Here, as specific examples of the application of the near view reaction measurement system 100, an application relating to quantitative recognition of functional degradation due to aging, an application relating to quantitative recognition of asthenopia, and an application relating to the detection of binocular vision abnormality will be described.

### (7-1) Quantitative Recognition of Functional Degradation Due to Aging

In the application relating to quantitative recognition of functional degradation due to aging, for example, the image processing device 1 calculates, as the near view reaction index, a pupil size in a far view state (that is, the maximum value Dmax of the pupil size), a pupil size in a near view state (that is, the minimum value Dmin of the pupil size or the pupil size after re-expansion), a pupil contraction speed, a convergence amount, a change speed of the convergence degree, and the like. Then, the image processing device 1 compares the calculated values of the various near view reaction indexes with the reference values of the near view reaction indexes, and quantitatively estimates the degree of functional degradation of the subject 6 due to aging. Then, the image processing device 1 outputs the estimation result to the output device 3. The reference value described above may be a standard value of various common near view reaction indexes at the age of the subject 6, or may be a past calculated value of the calculated value of the near view reaction index of the subject 6. The reference value described above may be a threshold for determining the presence or absence or level of functional degradation due to aging.

Here, the image processing device 1 may use a model for estimating the degree of functional degradation due to aging of the subject 6 and cause the output device 3 to output the information output by the model. The above-described model is a machine learning model such as an equation, a lookup table, or a neural network, and outputs, for example, an estimation result (for example, an estimated age of the subject 6) regarding a degree of functional degradation due to aging in a case where a calculated value of various near view reaction indexes (or a difference between the calculated value and a reference value) is used as an input. Parameters and the like of the above-described model are stored in advance in the storage device 4, the memory 12, or the like. The image processing device 1 may display a graph or the like showing the calculated values of various near view reaction indexes and the relevant reference values in a comparable manner.

According to such an application, the subject 6 can easily measure the near view reaction by the subject 6 himself/herself using his/her own smartphone or the like, and quantitatively grasp the functional degradation due to aging. Then, the image processing device 1 can also propose an activity of preventing functional degradation due to aging, visualize the effect of preventing functional degradation due to aging, and the like, and can help the user who is the subject 6 enhance the continuous consciousness of the preventive activity against functional degradation due to aging.

### (7-2) Quantitative Recognition of Asthenopia

In the application relating to quantitative recognition of asthenopia, the image processing device 1 calculates, for example, the maximum pupil contraction rate, the pupil contraction speed, the re-expansion speed, the convergence amount, and the like as the near view reaction index. Then, the image processing device 1 compares the calculated values of the various near view reaction indexes with the reference values of the near view reaction indexes, and quantitatively estimates the degree of asthenopia of the subject 6. Then, the image processing device 1 outputs the estimation result to the output device 3. The reference value described above may be a standard value of various common near view reaction indexes at the age of the subject 6, or may be a past calculated value of the near view reaction index of the subject 6. The reference value described above may be a threshold for determining the presence or absence or level of asthenopia of the subject 6.

Here, the image processing device 1 may use a model for estimating the degree of asthenopia of the subject 6 and cause the output device 3 to output the information output by the model. The above-described model is a machine learning model such as an equation, a lookup table, or a neural network, and outputs, for example, an estimation result regarding the degree of asthenopia of the subject 6 in a case where a calculated value of various near view reaction indexes (or a difference between the calculated value and a reference value) is used as an input. Parameters and the like of the above-described model are stored in advance in the storage device 4, the memory 12, or the like. The image processing device 1 may display, as the information indicating the degree of asthenopia of the subject 6, a graph or the like showing the calculated values of various near view reaction indexes and the relevant reference values in a comparable manner.

According to such an application, the subject 6 can easily measure the near view reaction by the subject 6 himself/herself using his/her own smartphone or the like, and quantitatively grasp the asthenopia.

### (7-3) Detection of Binocular Vision Abnormality

Next, an application relating to detection of binocular vision abnormality (including esotropia due to use of a smartphone) will be described.

In this application, the image processing device 1 calculates, for example, the maximum pupil contraction rate, the pupil contraction speed, the convergence amount, the change speed of the convergence degree, and the like as the near view reaction index. In the present application, the image processing device 1 calculates a far view reaction index in addition to a near view reaction index. Therefore, the image processing device 1 again performs the far view instruction by the first instruction unit 14 after the near view instruction by the second instruction unit 17, and calculates the far view reaction index based on the time-series first pupil information (and the immediately preceding second pupil information) generated by the first pupil information generation unit 15 after the far view instruction. As a result, the image processing device 1 calculates a pupil expansion rate, a pupil expansion speed, a convergence amount, a change speed of the convergence degree, and the like associated with the reaction of the subject 6 accompanying the switching from the near view state to the far view state. The “pupil expansion rate” is a ratio of the pupil size in the subsequent far view state to the pupil size in the near view state, and the “pupil expansion speed” is an expansion speed of the pupil size in an expansion period of the pupil size generated by switching from the near view state to the far view state.

Then, the image processing device 1 compares the calculated values of various near view reaction indexes and far view reaction indexes with reference values of various near view reaction indexes and far view reaction indexes, and estimates binocular vision abnormality of the subject 6. Then, the image processing device 1 outputs the estimation result to the output device 3. The reference value described above may be a standard value of various general near view reaction indexes and far view reaction indexes at the age of the subject 6, or may be a past calculated value of the near view reaction index and the far view reaction index of the subject 6. The reference value described above may be a threshold for determining the presence or absence or level of binocular vision abnormality of the subject 6.

In a case where the state is relevant to any one of a state in which the pupil does not constrict even if convergence occurs in the near view reaction and a state in which the convergence degree does not return to the initial convergence degree at the time of far view in the reaction of the subject 6 accompanying the switching from the near view state to the far view state, there is a high possibility that binocular vision abnormality occurs. Therefore, the above-described threshold may be set to be a standard value for determining whether the state is relevant to any of the above-described states.

According to such an application, the subject 6 can easily measure the near view reaction by the subject 6 himself/herself using his/her own smartphone or the like, and can grasp the determination result of the binocular vision abnormality. The image processing device 1 can also propose a preventive activity according to the determination result of binocular vision abnormality, visualize the preventive effect, and the like, and can help the subject 6 enhance the continuous consciousness of the preventive activity regarding binocular vision abnormality. This application can be used not only for self-checking of the subject 6 but also for checking by a parent in a case where the subject 6 is a minor.

### Second Example Embodiment

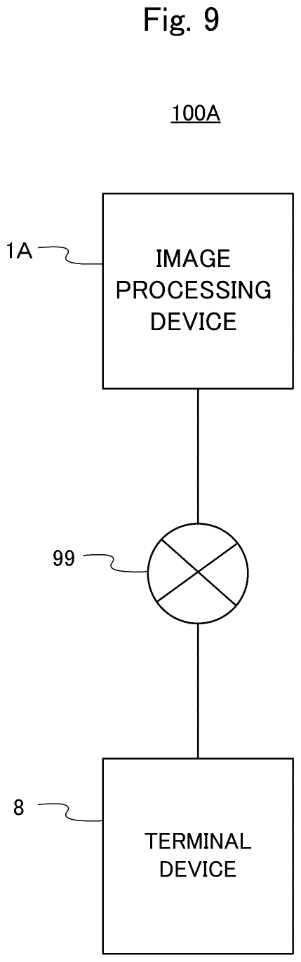

FIG. 9 illustrates a schematic configuration of a near view reaction measurement system 100A in a second example embodiment. The near view reaction measurement system 100A according to the second example embodiment includes an image processing device 1A that functions as a server and a terminal device 8 that is used by a subject and functions as a client. The image processing device 1A and the terminal device 8 perform data communication via a network 99. Hereinafter, the same components as those of the first example embodiment are appropriately denoted by the same reference signs, and description thereof will be omitted.

The terminal device 8 is a terminal used by a user as the subject, has an input function, a display function, a communication function, and an imaging function, and functions as the input device 2, the output device 3, the measurement device 5 including the camera 51, and the like illustrated in FIG. 1. The terminal device 8 may be, for example, a tablet terminal such as a personal computer or a smartphone, or a personal digital assistant (PDA). The terminal device 8 transmits the face image of the subject output by the camera 51 to the image processing device 1A via the network 99.

The image processing device 1A has the same hardware configuration as the hardware configuration of the image processing device 1 illustrated in FIG. 2, and the processor 11 of the image processing device 1A has the functional blocks illustrated in FIG. 4 described in the first example embodiment. Then, the image processing device 1A receives the camera image from the terminal device 8 via the network 99, and executes the near view reaction measurement processing of the subject. The image processing device 1A transmits an output signal for outputting a processing result to the terminal device 8 via the network 99 based on a display request from the terminal device 8.

As described above, the image processing device 1A in the second example embodiment performs processing relating to the measurement of the near view reaction of the subject who is the user of the terminal device 8, and can more suitably present the measurement result of the near view reaction to the subject by the terminal device 8.

### Third Example Embodiment

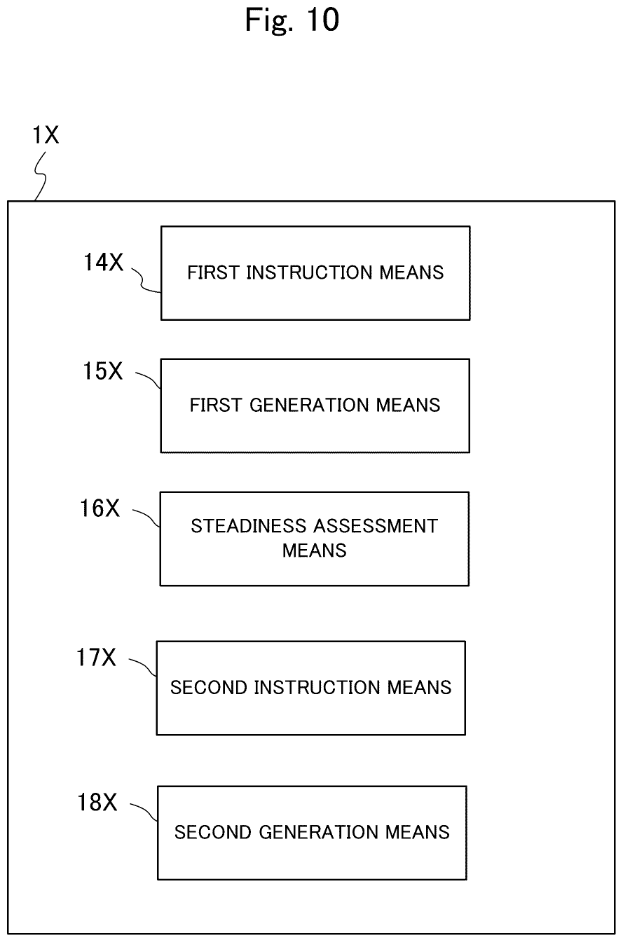

FIG. 10 is a block diagram of the image processing device 1X according to the third example embodiment. The image processing device 1X mainly includes the first instruction means 14X, first generation means 15X, a steadiness assessment means 16X, the second instruction means 157, and the second generation means 18X. The image processing device 1X may be configured by plural devices.

The first instruction means 14X is configured to instruct a subject to be in a first state in which the subject is looking at a position ahead by a first distance. Examples of the first instruction means 14X include the first instruction unit 14 according to the first or second example embodiment.

The first generation means 15X is configured to generate first pupil information that is information relating to a pupil of the subject based on an image generated by a photographing means in a case where the subject is in the first state. Examples of the first generation means 15X include the first pupil information generation unit 15 according to the first or second example embodiment.

The steadiness assessment means 16X is configured to determine presence or absence of steadiness of the pupil of the subject in the first state based on the first pupil information. Examples of the steadiness assessment means 16X include the steadiness assessment unit 16 according to the first or second example embodiment.

The second instruction means 17X is configured to instructing, upon determining the presence of the steadiness, the subject to be in a second state in which the subject is looking at a position ahead by a second distance shorter than the first distance. Examples of the second instruction means 17X include the second instruction unit 17 according to the first or second example embodiment.

The second generation means 18X is configured to generate second pupil information that is information relating to the pupil based on an image generated by the photographing means in a case where the subject is in the second state. Examples of the second generation means 18X include the second pupil information generation unit 18 according to the first or second example embodiment.

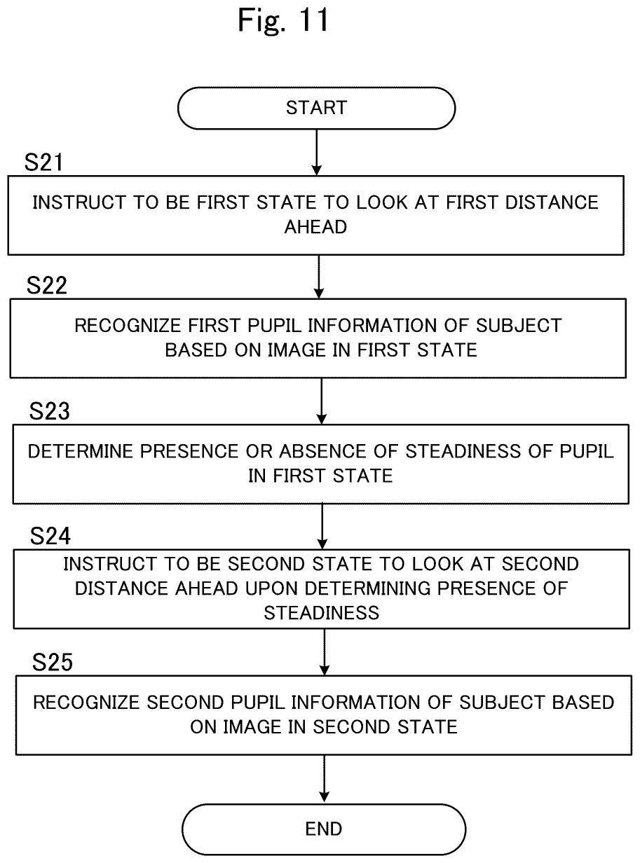

FIG. 11 illustrates an example of the flowchart executed by the image processing device 1X according to the third example embodiment. The first instruction means 14X instructs a subject to be in a first state in which the subject is looking at a position ahead by a first distance (step S21). The first generation means 15X generates first pupil information that is information relating to a pupil of the subject based on an image generated by a photographing means in a case where the subject is in the first state (step S22). The steadiness assessment means 16X determines presence or absence of steadiness of the pupil of the subject in the first state based on the first pupil information (step S23). The second instruction means 17X instructs, upon determining the presence of the steadiness, the subject to be in a second state in which the subject is looking at a position ahead by a second distance shorter than the first distance (step S24). The second generation means 18X generates second pupil information that is information relating to the pupil based on an image generated by the photographing means in a case where the subject is in the second state (step S25).

According to the third example embodiment, the image processing device 1X can suitably acquire pupil information regarding near view response of the subject.

In the example embodiments described above, the program is stored by any type of a non-transitory computer-readable medium (non-transitory computer readable medium) and can be supplied to a control unit or the like that is a computer. The non-transitory computer-readable medium include any type of a tangible storage medium. Examples of the non-transitory computer readable medium include a magnetic storage medium (e.g., a flexible disk, a magnetic tape, a hard disk drive), a magnetic-optical storage medium (e.g., a magnetic optical disk), CD-ROM (Read Only Memory), CD-R, CD-R/W, a solid-state memory (e.g., a mask ROM, a PROM (Programmable ROM), an EPROM (Erasable PROM), a flash ROM, a RAM (Random Access Memory)). The program may also be provided to the computer by any type of a transitory computer readable medium. Examples of the transitory computer readable medium include an electrical signal, an optical signal, and an electromagnetic wave. The transitory computer readable medium can provide the program to the computer through a wired channel such as wires and optical fibers or a wireless channel.

The whole or a part of the example embodiments described above (including modifications, the same applies hereinafter) can be described as, but not limited to, the following Supplementary Notes.

### [Supplementary Note 1]

An image processing device comprising:
a first instruction means for instructing a subject to be in a first state in which the subject is looking at a position ahead by a first distance;
a first generation means for generating first pupil information that is information relating to a pupil of the subject based on an image generated by a photographing means in a case where the subject is in the first state;
a steadiness assessment means for determining presence or absence of steadiness of the pupil of the subject in the first state based on the first pupil information;
a second instruction means for instructing, upon determining the presence of the steadiness, the subject to be in a second state in which the subject is looking at a position ahead by a second distance shorter than the first distance; and
a second generation means for generating second pupil information that is information relating to the pupil based on an image generated by the photographing means in a case where the subject is in the second state.

### [Supplementary Note 2]

The image processing device according to Supplementary Note 1, further comprising a near view reaction calculation means for calculating an index relating to a near view reaction of the subject based on the second pupil information.

### [Supplementary Note 3]

The image processing device according to Supplementary Note 2, wherein the near view reaction calculation means calculates the index based on the time-series second pupil information generated after an instruction to enter the second state is issued.

### [Supplementary Note 4]

The image processing device according to Supplementary Note 1, wherein the first pupil information and the second pupil information include information relating to a degree of convergence and a size of the pupil.

### [Supplementary Note 5]

The image processing device according to Supplementary Note 4, wherein
the image is an image obtained by photographing at least both eyes of the subject, and
the first generation means and the second generation means calculate the degree of convergence based on positions of pupils of the both eyes in the image.

### [Supplementary Note 6]

The image processing device according to Supplementary Note 1, wherein the steadiness assessment means determines presence or absence of steadiness of the pupil based on presence or absence of a change in a focal point, presence or absence of light reflection, and presence or absence of a change in a size of the pupil.

[supplementary Note 7]

The image processing device according to Supplementary Note 2, wherein
the first instruction means re-instructs the subject to enter the first state after generation of the second pupil information, and
the near view reaction calculation means calculates an index relating to a reaction of the subject in response to switching from the second state to the first state based on the first pupil information generated after the re-instruction.

### [Supplementary Note 8]

The image processing device according to Supplementary Note 2, wherein the near view reaction calculation means calculates, as the index, at least one of indexes relating to a change in a maximum value of a size of the pupil, a minimum value of the size, a maximum contraction rate of the pupil, a contraction speed of the pupil, a re-expansion speed of the pupil, or a degree of convergence.

### [Supplementary Note 9]

The image processing device according to Supplementary Note 2, wherein the near view reaction calculation means outputs information relating to a state of the subject estimated based on the index by an output device.

### [Supplementary Note 10]

The image processing device according to Supplementary Note 9, wherein the near view reaction calculation means outputs a degree of functional degradation due to aging of the subject estimated based on the index as information relating to the state.

### [Supplementary Note 11]

The image processing device according to Supplementary Note 9, wherein the near view reaction calculation means outputs a degree of asthenopia of the subject estimated based on the index as information relating to the state.

### [Supplementary Note 12]

The image processing device according to Supplementary Note 9, wherein the near view reaction calculation means outputs information relating to binocular vision abnormality of the subject estimated based on the index as information relating to the state.

### [Supplementary Note 13]

An image processing method executed by a computer, comprising:
instructing a subject to be in a first state in which the subject is looking at a position ahead by a first distance;
generating first pupil information that is information relating to a pupil of the subject based on an image generated by a photographing means in a case where the subject is in the first state;
determining presence or absence of steadiness of the pupil of the subject in the first state based on the first pupil information;
instructing, upon determining the presence of the steadiness, the subject to be in a second state in which the subject is looking at a position ahead by a second distance shorter than the first distance; and
generating second pupil information that is information relating to the pupil based on an image generated by the photographing means in a case where the subject is in the second state.

### [Supplementary Note 14]

A storage medium storing a program for causing a computer to perform a process comprising:
instructing a subject to be in a first state in which the subject is looking at a position ahead by a first distance;
generating first pupil information that is information relating to a pupil of the subject based on an image generated by a photographing means in a case where the subject is in the first state;
determining presence or absence of steadiness of the pupil of the subject in the first state based on the first pupil information;
instructing, upon determining the presence of the steadiness, the subject to be in a second state in which the subject is looking at a position ahead by a second distance shorter than the first distance; and
generating second pupil information that is information relating to the pupil based on an image generated by the photographing means in a case where the subject is in the second state.

While the invention has been particularly shown and described with reference to example embodiments thereof, the invention is not limited to these example embodiments. It will be understood by those of ordinary skilled in the art that various changes in form and details may be made therein without departing from the spirit and scope of the present invention as defined by the claims. In other words, it is needless to say that the present invention includes various modifications that could be made by a person skilled in the art according to the entire disclosure including the scope of the claims, and the technical philosophy. All Patent and Non-Patent Literatures mentioned in this specification are incorporated by reference in its entirety.

### DESCRIPTION OF REFERENCE NUMERALS

1, 1A, 1X Image processing device
2 Input device
3 Output device
4 Storage device
5 Measurement device
8 Terminal device
11 Processor
12 Memory
13 Interface
51 Camera
90 Data bus
99 Network
100, 100A Near view reaction measurement system

What is claimed is:

1. An image processing device comprising:
at least one memory configured to store instructions; and
at least one processor configured to execute the instructions to:
instruct a subject to be in a first state in which the subject is looking at a position ahead by a first distance;
generate first pupil information that is information relating to a pupil of the subject based on an image generated by a photographing the subject in a case where the subject is in the first state;
determine presence or absence of steadiness of the pupil of the subject in the first state based on the first pupil information;
instruct, upon determining the presence of the steadiness, the subject to be in a second state in which the subject is looking at a position ahead by a second distance shorter than the first distance; and
generate second pupil information that is information relating to the pupil based on an image generated by the photographing the subject in a case where the subject is in the second state.

2. The image processing device according to claim 1, comprising wherein the at least one processor is configured to execute the instructions to calculate an index relating to a near view reaction of the subject based on the second pupil information.

3. The image processing device according to claim 2, wherein the at least one processor is configured to execute the instructions to calculate the index based on the time-series second pupil information generated after an instruction to enter the second state is issued.

4. The image processing device according to claim 1, wherein the first pupil information and the second pupil information include information relating to a degree of convergence and a size of the pupil.

5. The image processing device according to claim 4, wherein
the image is an image obtained by photographing at least both eyes of the subject, and
the at least one processor is configured to execute the instructions to calculate the degree of convergence based on positions of pupils of the both eyes in the image.

6. The image processing device according to claim 1, wherein the at least one processor is configured to execute the instructions to determine presence or absence of steadiness of the pupil based on presence or absence of a change in a focal point, presence or absence of light reflection, and presence or absence of a change in a size of the pupil.

7. The image processing device according to claim 2, wherein
the at least one processor is configured to execute the instructions to
re-instruct the subject to enter the first state after generation of the second pupil information, and
calculate an index relating to a reaction of the subject in response to switching from the second state to the first state based on the first pupil information generated after the re-instruction.

8. The image processing device according to claim 2, wherein the at least one processor is configured to execute the instructions to calculate, as the index, at least one of indexes relating to a change in a maximum value of a size of the pupil, a minimum value of the size, a maximum contraction rate of the pupil, a contraction speed of the pupil, a re-expansion speed of the pupil, or a degree of convergence.

9. The image processing device according to claim 2, wherein the at least one processor is configured to execute the instructions to output information relating to a state of the subject estimated based on the index by an output device.

10. The image processing device according to claim 9, wherein the at least one processor is configured to execute the instructions to output degree of functional degradation due to aging of the subject estimated based on the index as information relating to the state.

11. The image processing device according to claim 9, wherein the at least one processor is configured to execute the instructions to output degree of asthenopia of the subject estimated based on the index as information relating to the state.

12. The image processing device according to claim 9, wherein the at least one processor is configured to execute the instructions to output information relating to binocular vision abnormality of the subject estimated based on the index as information relating to the state.

13. An image processing method executed by a computer, comprising:
instructing a subject to be in a first state in which the subject is looking at a position ahead by a first distance;
generating first pupil information that is information relating to a pupil of the subject based on an image generated by a photographing the subject in a case where the subject is in the first state;
determining presence or absence of steadiness of the pupil of the subject in the first state based on the first pupil information;
instructing, upon determining the presence of the steadiness, the subject to be in a second state in which the subject is looking at a position ahead by a second distance shorter than the first distance; and
generating second pupil information that is information relating to the pupil based on an image generated by the photographing the subject in a case where the subject is in the second state.

14. A non-transitory computer readable storage medium storing a program for causing a computer to perform a process comprising:
instructing a subject to be in a first state in which the subject is looking at a position ahead by a first distance;
generating first pupil information that is information relating to a pupil of the subject based on an image generated by a photographing the subject in a case where the subject is in the first state;
determining presence or absence of steadiness of the pupil of the subject in the first state based on the first pupil information;
instructing, upon determining the presence of the steadiness, the subject to be in a second state in which the subject is looking at a position ahead by a second distance shorter than the first distance; and
generating second pupil information that is information relating to the pupil based on an image generated by photographing the subject in a case where the subject is in the second state.
